(19) United States
(12) Patent Application Publication
(10) Pub. No.: US 2013/0261493 A1
(43) Pub. Date: Oct. 3, 2013

(54) BIO-PROBE ASSEMBLY

(71) Applicant: CHUNG HUA UNIVERSITY, Hsinchu (TW)

(72) Inventor: Jium Ming LIN, HSINCHU (TW)

(73) Assignee: CHUNG HUA UNIVERSITY, Hsinchu (TW)

(21) Appl. No.: 13/854,388

(22) Filed: Apr. 1, 2013

(30) Foreign Application Priority Data
Apr. 2, 2012 (TW) 101111629

(51) Int. Cl. A61B 5/053

(52) U.S. Cl. CPC A61B 5/0532
USPC 600/548; 600/554

(57) ABSTRACT

A bio-probe assembly comprises a flexible substrate, a probe array, and an adhesive. The flexible substrate has a surface. The probe array has a plurality of probes, which is disposed on the surface of the flexible substrate. The adhesive is disposed on the surface of the flexible substrate as well. Each probe has a protrusion length in a range from 100 to 300 micrometers.

### BACKGROUND OF THE INVENTION

1. Technical Field

The present invention relates to a bio-probe assembly for detecting electrical properties of acupuncture points or providing signals to acupuncture points.

2. Background

In the 1950s, when Dr. Reinhard Voll studied acupuncture points, he discovered that there are nearly 2000 acupuncture points on the skin surface, and the acupuncture points are distributed along channels, also called meridians. The acupuncture points are specific superficial anatomic locations, which have electrical resistances lower than those of their surrounding skin. In his studies, Dr. Voll further discovered that the measured resistance data could be used to assess the health statuses of corresponding organs. With such a discovery, electro-therapy methods were developed by introducing therapy signals to acupuncture points through electrodes to treat corresponding organs.

U.S. Pat. No. 4,981,146, U.S. Pat. No. 5,397,338, U.S. Pat. No. 5,626,617, U.S. Pat. No. 6,735,480, and U.S. Patent Publication No. 2005/0197555 are all related to the therapeutic or health-monitoring techniques regarding the acupuncture points. Traditionally, the resistance or impedance of an acupuncture point is detected using a bio-impedance measuring device and a metal probe. In every measurement, the probe can only measure acupuncture points. The device operator or therapist must require a certain level of knowledge and experience in acupuncture points in order to precisely perform measurements or apply therapy signals. An inexperienced user cannot utilize such a scientific discovery at home.

When an operator uses a conventional bio-impedance measuring device, the operator should press the device's probe on the skin of a treated object by his hand. However, a consistent application of pressure cannot guarantee in different measurements, which might result in poor electrical contacts and measurement results. In addition, if too much pressure is applied, the treated object or testee will have an unpleasant experience.

### SUMMARY

In view of the above issues, the present invention proposes new bio-probe assemblies.

One embodiment of the present invention discloses a bio-probe assembly, which comprises a flexible substrate, a probe array, and an adhesive. The flexible substrate comprises a surface. The probe array comprises a plurality of probes, which are arranged on the surface of the flexible substrate. Each probe comprises a protrusion length in a range from 100 to 300 micrometers. The adhesive is disposed on the surface of the flexible substrate.

Another embodiment of the present invention discloses a bio-probe assembly, which comprises a flexible substrate, a first adhesive, a probe array, and a second adhesive. The flexible substrate has a surface. The probe array comprises a plurality of probes, wherein the first adhesive fastens the probe array on the surface of the flexible substrate, and each probe comprises a protrusion length in a range from 100 to 300 micrometers. The second adhesive is disposed on the surface of the flexible substrate.

The foregoing has outlined rather broadly the features and technical advantages of the present invention in order that the detailed description of the invention that follows may be better understood. Additional features and advantages of the invention will be described hereinafter, and form the subject of the claims of the invention. It should be appreciated by those skilled in the art that the conception and specific embodiment disclosed may be readily utilized as a basis for modifying or designing other structures or processes for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

The objectives and advantages of the present invention are illustrated with the following description and upon reference to the accompanying drawings in which:

FIG. 1 schematically shows a bio-probe system according to one embodiment of the present invention;

FIG. 2 is a top view of a bio-probe assembly according to one embodiment of the present invention;

FIG. 3 is a cross-sectional view along line 1-1 of FIG. 2;

FIG. 4 schematically shows the probe array of a bio-probe assembly according to one embodiment of the present invention;

FIG. 5 schematically shows a bio-probe assembly being attached to a finger according to one embodiment of the present invention;

FIG. 6 is a top view of a bio-probe assembly according to another embodiment of the present invention;

FIG. 7 is a cross-sectional view along line 2-2 of FIG. 6;

FIG. 8 schematically shows a patterned polysilicon layer on a flexible substrate formed in a step of a method for forming a bio-probe assembly according to one embodiment of the present invention;

FIG. 9 is a cross-sectional view along line 3-3 of FIG. 8;

FIG. 10 is a top view of a structure formed after the unwanted portions of the chrome, nickel and gold layers are removed by a lift-off process according to one embodiment of the present invention;

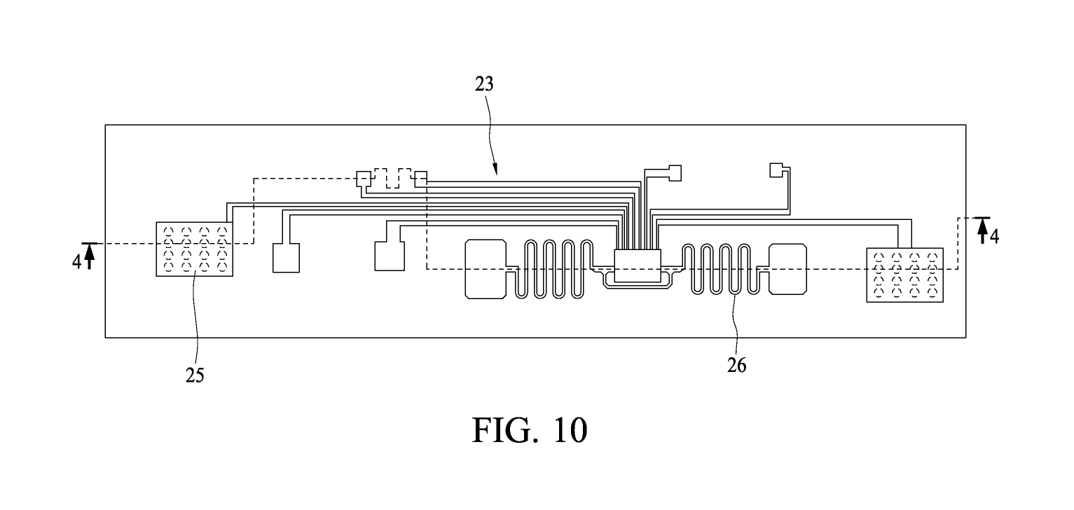

FIG. 11 is a cross-sectional view along line 4-4 of FIG. 10;

FIG. 12 is a cross-sectional view of a bio-probe assembly according to one embodiment of the present invention;

FIG. 13 schematically shows probe arrays formed on an electrically conductive tape according to one embodiment of the present invention; and

FIG. 14 schematically shows a thin film capacitor according to one embodiment of the present invention.

### DETAILED DESCRIPTION

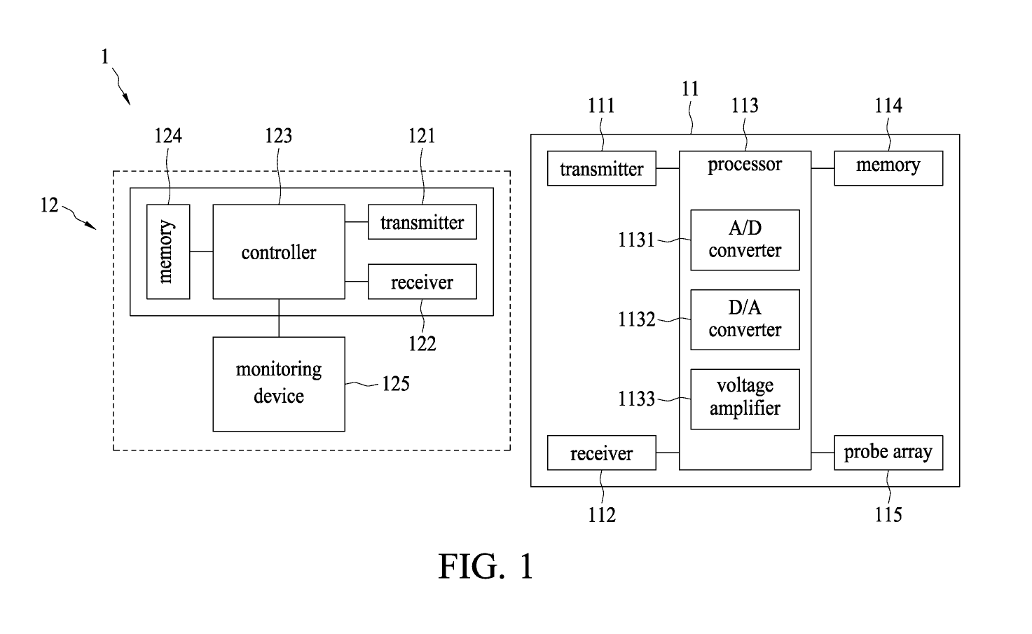

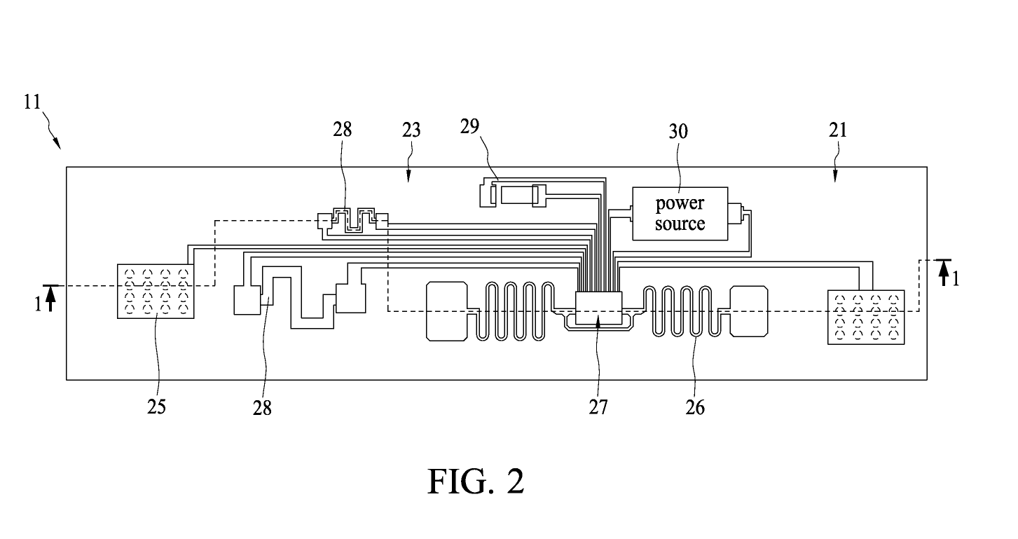

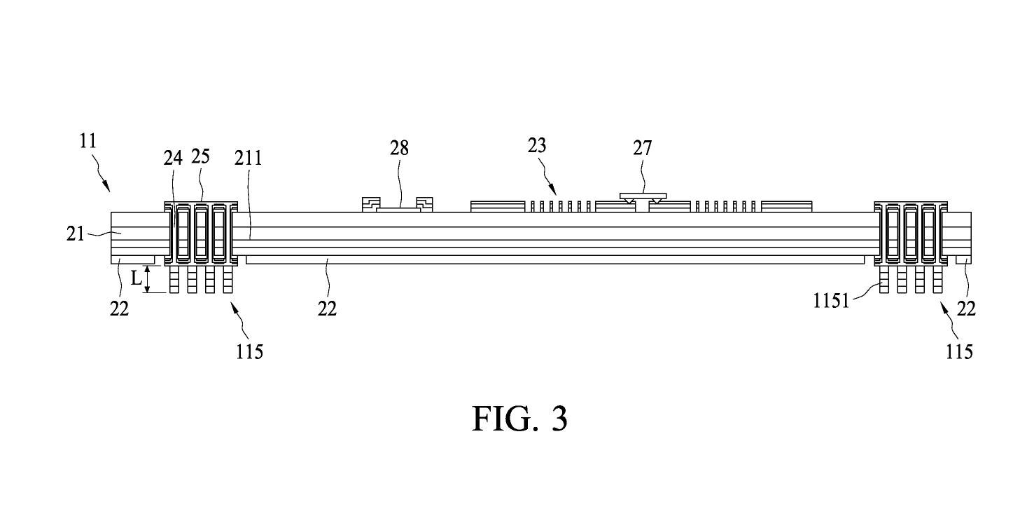

FIG. 1 schematically shows a bio-probe system 1 according to one embodiment of the present invention. FIG. 2 is a top view of a bio-probe assembly 11 according to one embodiment of the present invention. FIG. 3 is a cross-sectional view along line 1-1 of FIG. 2. Referring to FIGS. 1 to 3, the bio-probe system 1 is configured to detect the electrical properties of acupuncture points and carries on treatment through the acupuncture points. The bio-probe system 1 comprises a bio-probe assembly 11 and a host 12. The bio-probe assembly 11 may comprise at least one probe array 115, each of which is configured to contact the skin where an acupuncture point is located to perform measurements or provide a therapy signal. In some embodiments, the bio-probe assembly 11 may comprise a plurality of probe arrays 115. The host 12 can transmit a code to the bio-probe assembly 11, and the code can enable the bio-probe assembly 11 to provide a suitable therapy signal, or enable the bio-probe assembly 11 to measure the electrical property, such as impedance, and an electrical potential. In some embodiments, the code may comprise a code segment, which is used for determining the code's related acupuncture point. In some embodiments, the code comprises the information regarding the strength of a therapy signal. In some embodiments, the code may comprise the frequency of a therapy signal. In addition, the bio-probe assembly 11 may be configured to transmit the measured data of an acupuncture point to the host 12. The host 12 can evaluate the health status of an organ corresponding to the acupuncture point or assess the effect after treatment with a therapy signal by using the measured data. The host 12 can transmit, according to the measured data, a new code to change the strength or frequency of the therapy signal in use.

Preferably, the host 12 and the bio-probe assembly 11 can communicate with each other through wireless technology. In some embodiments, the host 12 and the bio-probe assembly 11 can communicate with each other by RFID (Radio Frequency Identification) protocol. In some embodiments, the host 12 and the bio-probe assembly 11 can communicate with each other by another wireless communication protocol such as ZIGBEE or Bluetooth. In some embodiments, the host 12 and the bio-probe assembly 11 can communicate with each other through a wire.

Referring to FIG. 1, the host 12 may comprise a transmitter 121 and a receiver 122. The transmitter 121 is configured to modulate a code or a command and transmit the modulated code or command to at least one bio-probe assembly 11. The transmitter 121 can perform PWM (pulse width modulation), PIM (pulse interval modulation), or the like. The receiver 122 can receive modulated measured data from the bio-probe assembly 11 and demodulate the modulated measured data. The receiver 122 can perform amplitude shift keying (ASK) demodulation, phase shift keying (PSK) demodulation, or the like.

The host 12 may further comprise a controller 123 and a memory 124. The controller 123 is configured to control a transmitter 121, a receiver 122, and the memory 124. The controller 123 can provide a code or command to the transmitter 121 and receive data from the receiver 122. The controller 123 can store the received data in the memory 124 or transmit the received data to a monitoring device 125.

The monitoring device 125 can store the measured data of acupuncture points, and is configured to determine whether the measured data is within a normal range. The monitoring device 125 may be configured to provide a code to the controller 123. The monitoring device 125 may be configured to provide a new code to the controller 123 according to the measured data of an acupuncture point. The monitoring device 125 may be configured to modify the portion of a code that is used to generate a therapy signal by a trend of measured data, thereby allowing the bio-probe assembly 11 to generate a therapy signal with a different frequency or strength. The monitoring device 125 can obtain the received data from the receiver 122, determine which acupuncture point is related to the received data, and compare the received data with a standard reference one for the corresponding acupuncture point. The monitoring device 125 can store the data measured from each acupuncture point by a corresponding bio-probe assembly 11 for analysis, and the data can be printed out as a report.

The bio-probe assembly 11 may comprise a transmitter 111 and a receiver 112. The receiver 112 can be configured to demodulate a received modulated code or command The transmitter 111 can be configured to modulate data to be transmitted. The receiver 112 can perform ASK demodulation, PSK demodulation, or the like. The processor 113 can be configured to control the transmitter 111, the receiver 112, a memory 114, and the probe array 115. The processor 113 can generate a corresponding therapy signal or a stimulating current using a received code or command The memory 114 can store the operating programs for the bio-probe assembly 11, the necessary operating data, and the generated data. For example, the memory 114 can store the data to be transmitted, the data measured from an acupuncture point, or the information about which acupuncture point is being measured by the bio-probe assembly 11.

Referring to FIG. 1 again, the processor 113 may comprise an A/D (analog-to-digital) converter 1131, a D/A (digital-to-analog) converter 1132, and a voltage amplifier 1133. The D/A converter 1132 is configured to generate a stimulating current for an acupuncture point. The voltage amplifier 1133 is configured to amplify a response signal detected from the acupuncture point after the acupuncture point is stimulated with the stimulating current. The A/D converter 1131 is configured to convert the response signal to a digital response signal.

Referring to FIGS. 2 and 3, the bio-probe assembly 11 comprises a flexible substrate 21, at least one probe array 115, and an adhesive 22. The flexible substrate 21 has a surface 211. The at least one probe array 115 is arranged on the surface 211 of the substrate 21, and each probe array 115 comprises a plurality of probes 1151, each of which has a protrusion length L in a range from 100 to 300 micrometers. The adhesive 22 is disposed on the surface 211 of the substrate 21 and configured to connect the bio-probe assembly 11 with the skin

The flexible substrate 21 comprises polymer material. In some embodiments, the flexible substrate 21 comprises plastic material such as polyethylene terephthalate (PET) and polyimide (PI).

The probes 1151 can be arranged in a matrix. The probe array 115 can occupy approximately 0.25 square centimeters. The protrusion length of the probe 1151 can be less than 300 micrometers, preferably between 100 micrometers and 300 micrometers. The probe 1151 can have a non-sharpened tip to contact the skin surface but not to be inserted into the skin. The probe 1151 can be made of a biocompatible metal. The probe 1151 can comprise an electrically conductive mixture. In some embodiments, the probe 1151 can comprise polymer and silver.

The adhesive 22 can be an insulation adhesive. The adhesive 22 can be an adhesive that can be used on the skin. The adhesive 22 can be a medical grade adhesive. The adhesive 22 can comprise a removable adhesive. In some embodiments, the adhesive 22 can be included in a double-sided tape.

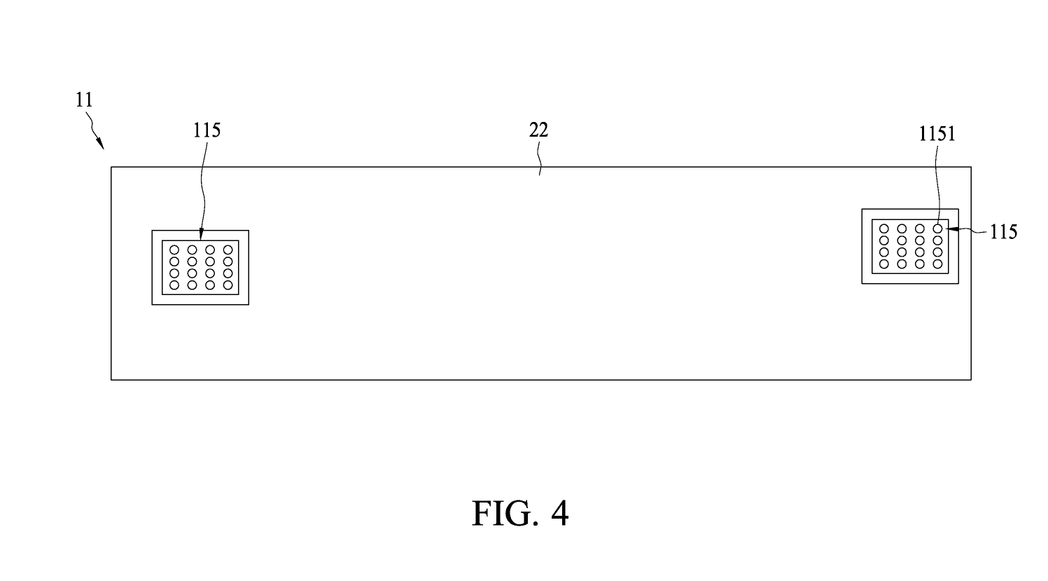

In some embodiments, the adhesive 22 can be applied to cover the entire surface 211 of the flexible substrate 21, except for the area where the probe array 115 is located as shown in FIG. 4.

Referring to FIGS. 2 and 3, in some embodiments, the bio-probe assembly 11 can further comprise a circuit 23. The circuit 23 can be formed on the surface 211 of the flexible substrate 21 and electrically connects to the probe array 115. In some embodiments, the probe array 115 electrically connects to the circuit 23 through at least one conductive pillar 24 formed through the flexible substrate 21. In some embodiments, the circuit 23 comprises a pad 25, which is configured to connect to the at least one conductive pillar 24.

Referring to FIGS. 1 and 2, the bio-probe assembly 11 may comprise a chip 27. The chip 27 can be coupled with the circuit 23 and is configured to include the transmitter 111, the receiver 112, the processor 113, and the memory 114. The circuit may comprise an antenna 26, which may be coupled with the chip 27.

In some embodiments, the circuit 23 may further comprise at least one thin film resistor 28. In some embodiments, the thin film resistor 28 can be an externally connected resistor of the chip 27.

In some embodiments, the circuit 23 can further comprise a thin film capacitor 29. In some embodiments, the thin film capacitor 29 can be an externally connected capacitor of the chip 27.

In some embodiments, the bio-probe assembly 11 can comprise a power source 30, which is configured to supply the power that the bio-probe assembly 11 can properly operate. In some embodiments, the power source 30 comprises a battery.

In some embodiments, the bio-probe assembly 11 is an RFID device, which can further comprise a rectifying module and an oscillating module. The rectifying module can be configured to convert microwave energy signals received by the antenna into electricity for supporting the operation of the bio-probe assembly 11 under passive mode operation. The oscillating module can generate a clock signal. In some embodiments, the rectifying module is coupled with the thin film capacitor 29. In some embodiments, the oscillating module comprises the thin film capacitor 29 and the thin film resistor 28. In some embodiments, the oscillating module can be a multi-vibrator.

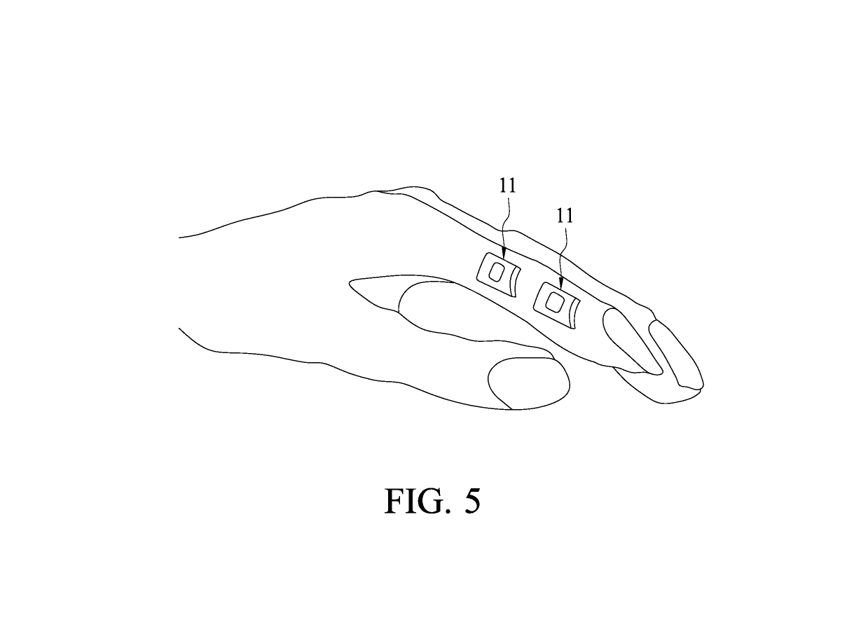

Referring to FIGS. 3 and 5, the bio-probe assembly 11 is bendable and can be attached to the skin using the adhesive 22 as shown in FIGS. 3 and 4. Due to the suitable protrusion lengths of the probes 1151 of the probe array 115, the probes 1151 can exert consistent pressure on the skin of different treated objects after the bio-probe assembly 11 is attached. As such, a better contact and a stable electrical connection between the probes 1151 and the skin can be ensured. The probe 1151 has a suitable protrusion length so that the testee or the treated object will not experience discomfort. In addition, a plurality of arrayed probes 1151 allows a user to perform measurements even if he does not know the exact position of the acupuncture point that he would like to measure.

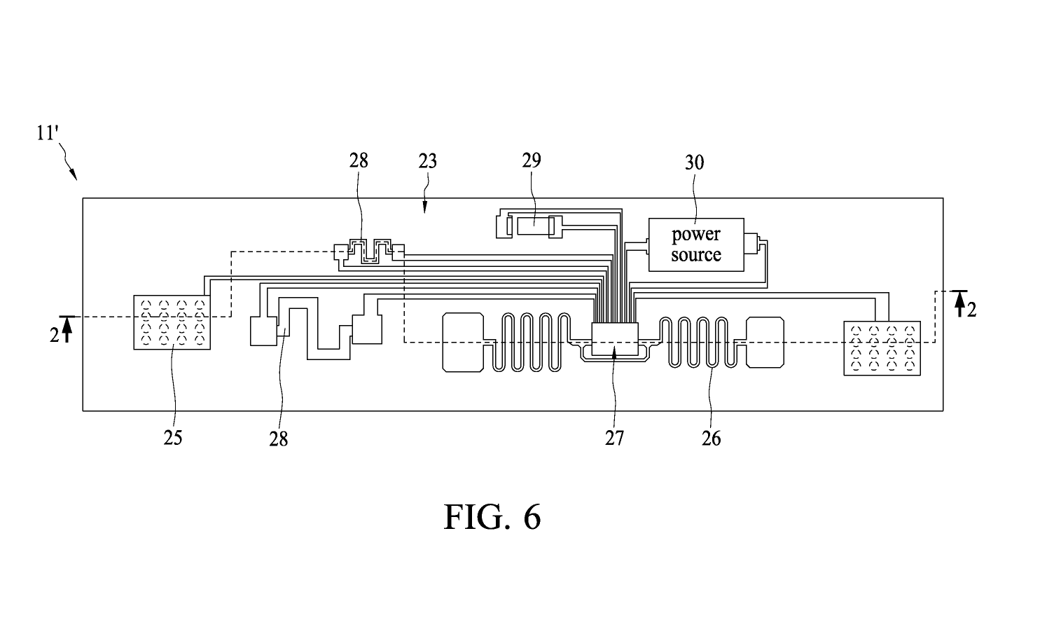

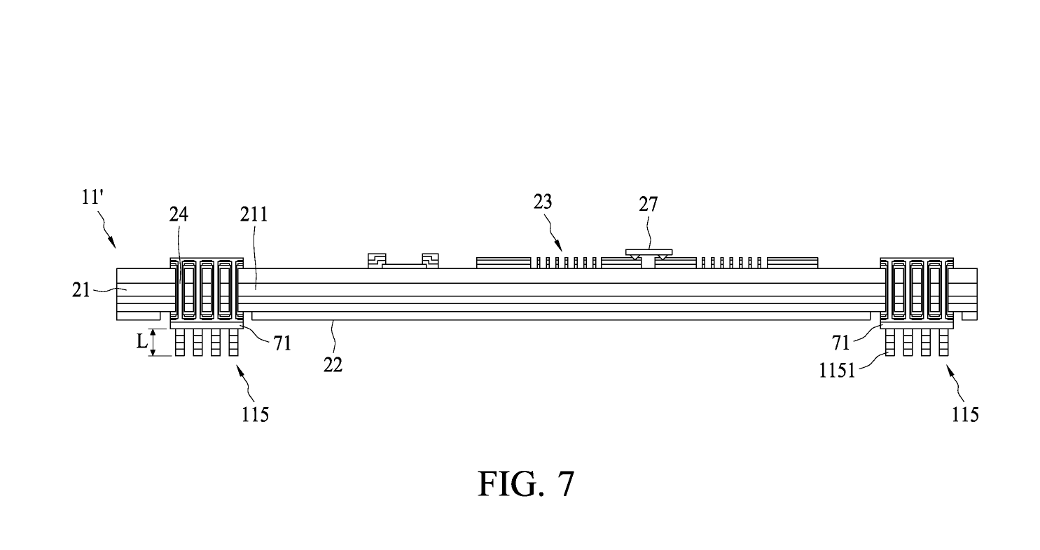

FIG. 6 is a top view of a bio-probe assembly 11′ according to another embodiment of the present invention, wherein the bio-probe assembly 11′ comprises a capacitor 29. FIG. 7 is a cross-sectional view along line 2-2 of FIG. 6. Referring to FIGS. 2, 3, 6, and 7, the bio-probe assembly 11′ is similar to the bio-probe assembly 11 disclosed in FIGS. 2 and 3, except that the probe array 115 of the bio-probe assembly 11′ is replaceable. In order to make the probe array 115 replaceable, in some embodiments, the bio-probe assembly 11′ comprises an adhesive 71 used to attach the probe array 115 to the flexible substrate 21. In some embodiments, the probe array 115 can be removed after the adhesive 71 is heated up. In some embodiments, the adhesive 71 can be electrically conductive. The probe array 115 electrically connects to the circuit 23 through the electrically conductive adhesive 71. In some embodiments, the adhesive 71 comprises a removable adhesive. In some embodiments, the adhesive 71 comprises a removable conductive adhesive. In some embodiments, the bio-probe assembly 11′ comprises an electrically conductive tape, which comprises a first conductive adhesive and a second conductive adhesive, wherein the first conductive adhesive is adhered to the surface 211 of the flexible substrate 21 and the second conductive adhesive is the adhesive 71.

The following illustratively demonstrates a method of manufacturing a bio-probe assembly; however, the present invention is not limited to such a demonstrated disclosure.

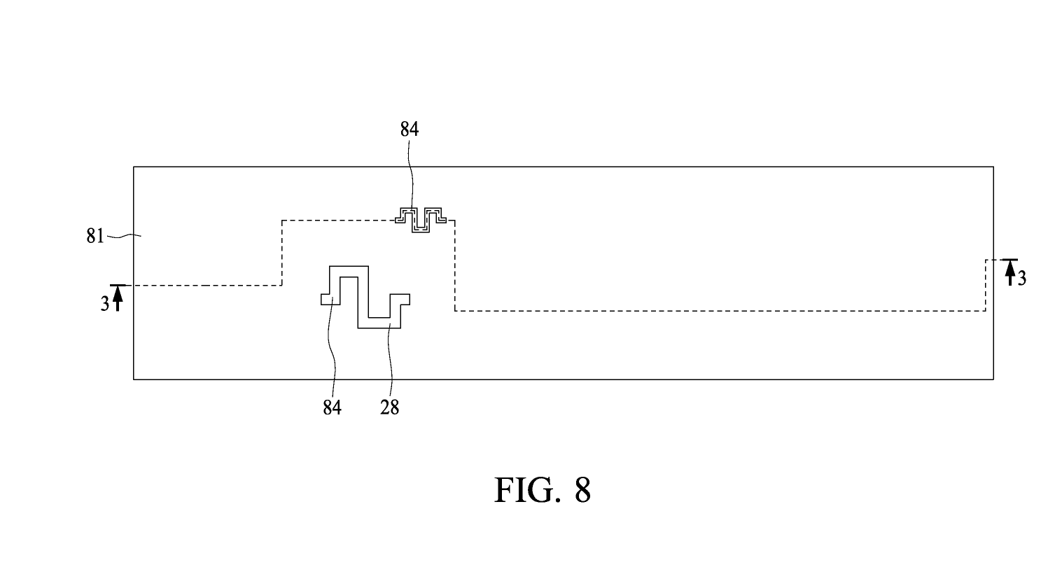

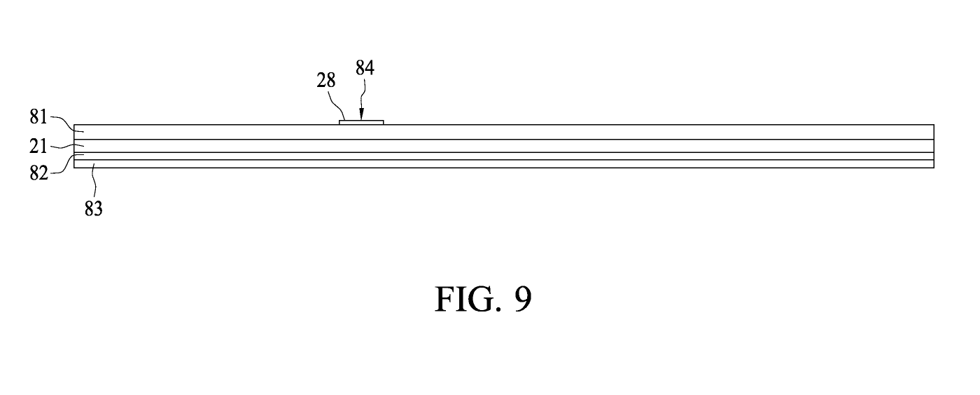

Referring to FIGS. 8 and 9, silicon dioxide layers 81 and 82 are respectively formed on two opposite surfaces of a flexible substrate 21. In some embodiments, the silicon dioxide layer 81 or 82 can have a thickness in a range from 1 to 10 micrometers. In some embodiments, the silicon dioxide layer 81 or 82 can be formed by a deposition method.

A protection layer 83 is formed on the silicon dioxide layer 82, wherein the protection layer 83 can protect the silicon dioxide layer 82 and prevent the silicon dioxide layer 82 from being affected by moisture. In some embodiments, the protection layer 83 comprises a photoresist. In some embodiments, the protection layer 83 comprises a positive photoresist. In some embodiments, the protection layer 83 can have a thickness from 0.5 to 10 micrometers.

An amorphous silicon layer 84 including p-type or n-type impurities are formed on the silicon dioxide layer 81 using a vapor-deposition method. The amorphous silicon layer 84 is then laser-annealed to form a polysilicon layer including p-type or n-type impurities. The polysilicon layer is then patterned to form a thin film resistor 28. In some embodiments, the amorphous silicon layer 84 is formed by vapor-depositing a powder mixture of p-type or n-type impurities and silicon. In some embodiments, the amorphous silicon layer 84 can have a thickness from 1 to 25 micrometers.

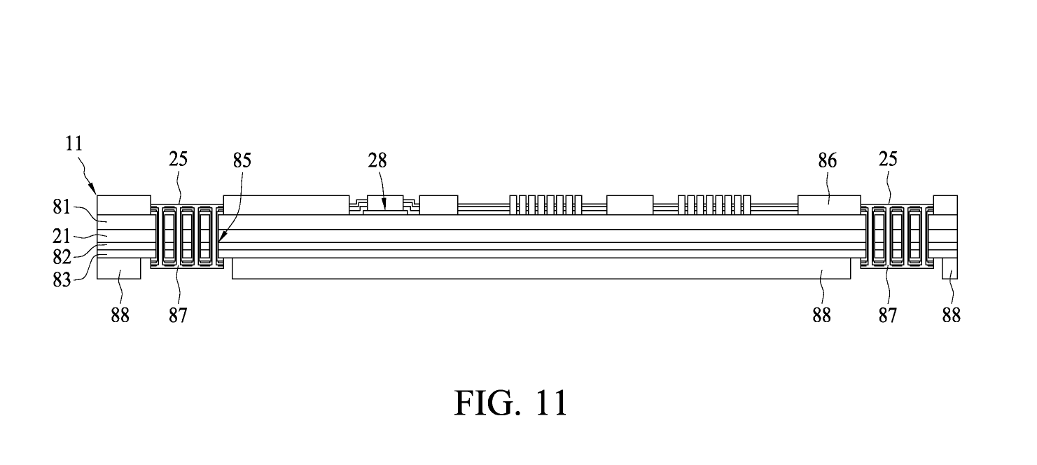

Referring to FIG. 11, a plurality of through-holes 85 are formed on the flexible substrate 21. In some embodiments, a laser can be used to form the through-holes 85.

Thereafter, chrome and nickel are sequentially deposited, and then a gold layer is formed by an electroless plating process. In particular, because the through-hole 85 has a large diameter and the flexible substrate 21 is thin, the through-holes 85 can be filled with chrome, nickel and gold. Subsequently, a photolithographic process is applied. Unwanted portions of metal layers are removed through a photoresist mask. After the photoresist mask is removed, the circuit 23 and the through-hole pillars can be obtained.

In other embodiments, after a plurality of through-hole 85 is formed on the flexible substrate 21, patterned photoresist layers 86 and 88 are respectively formed on the two surfaces of the flexible substrate 21. In some embodiments, the photoresist layers 86 and 88 comprise an SU-8 photoresist. Next, chrome and nickel are sequentially deposited, and then a gold layer is formed by an electroless plating process. Thereafter, the unwanted portions of the chrome, nickel and gold layers are removed by a lift-off process. Finally, the photoresist layers 86 and 88 are removed.

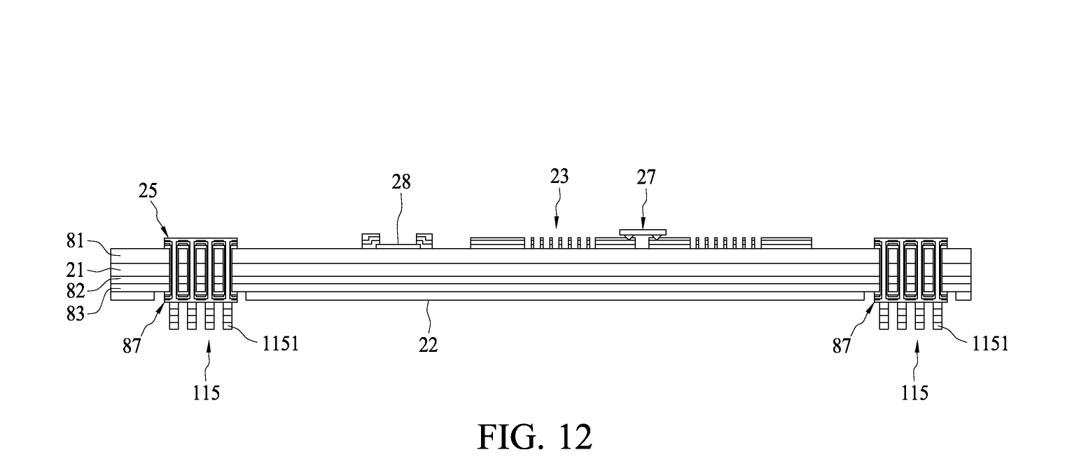

As shown in FIG. 12, probes 1151 are directly formed on pads including chrome, nickel and gold by a print-screening process. In some embodiments, the print-screening process can be repeatedly applied to form a probe 1151 having a plurality of stacking segments. In some embodiments, the print-screening process can be repeatedly applied until a probe 1151 has a protrusion length in a range from 100 to 300 micrometers. The probe 1151 having a protrusion length in a range from 100 to 300 micrometers can exert a suitable contact pressure on the skin, thereby achieving a better electrical connection effect. In some embodiments, a mixture of polymer and silver or a conductive material, such as electrically conductive polymer, is screen-printed to form the probes 1151.

Moreover, an adhesive 22 for attaching the bio-probe assembly is applied to the surface on the flexible substrate, except for the area where the probes 1151 are located. In some embodiments, the adhesive 22 comprises a removable adhesive. In some embodiments, the adhesive 22 is applied to cover the whole area outside the area where the probes 1151 are located. In some embodiments, a double-sided tape is stuck onto the protection layer 83, wherein the exposed adhesive is used as the adhesive 22. In some embodiments, the adhesive 22 is an insulation adhesive.

Furthermore, metal bumps are formed on the bonding pads of the circuit 23 by, for example, screen-printing an electrically conductive adhesive. The chip 27 can be flip-chip bonded to the corresponding bonding pads and fastened to the bonding pads by a thermal compression method. An underfill is formed to firmly attach the chip to the substrate 21 so that the chip may not be easily disconnected from the substrate 21. In addition, a battery socket is soldered onto the substrate 21, a battery is installed into the socket, and the bio-probe assembly is fabricated finally.

In other embodiments, the probe array 115 is removable or replaceable, and the manufacturing process is demonstrated as follows.

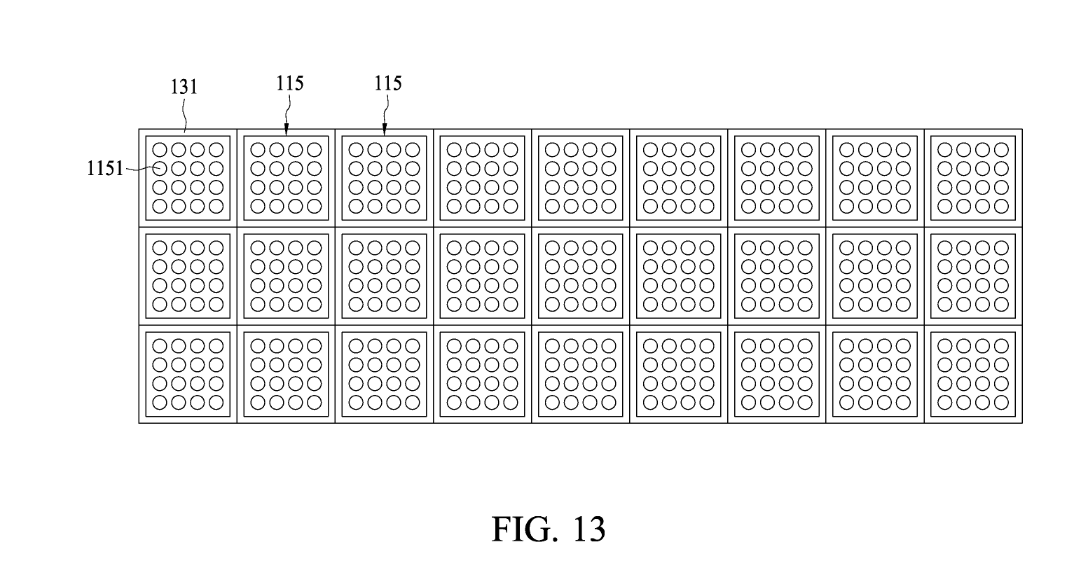

Referring to FIG. 13, a plurality of groups of probe arrays 115 is formed on an electrically conductive tape 131 by a screen-printing process. In some embodiments, the screen-printing process is repeated throughout a number of times to form probes 1151 with a protrusion length in a range from 100 to 300 micrometers. In some embodiments, the electrically conductive tape 131 can be a conductive silver tape or a conductive mesh. In some embodiments, the electrically conductive tape 131 can be a copper or aluminum foil tape. In some embodiments, the substrate of the electrically conductive tape 131 comprises copper, aluminum or an insulation material.

Furthermore, the electrically conductive tape 131 is cut into pieces. Then, after the photoresist layer 86 as shown in the embodiment of FIG. 11 is removed, the probe array 115 can be directly placed onto a pad 87 by the electrically conductive tape 131. Finally, the dispositions of the adhesive 22, the chip 27, the battery socket, and the battery are completed one after another. Due to the electrically conductive tape 131, the probe array 115 becomes replaceable. If the probe array 115 becomes dirty, the probe array 115 can be replaced. Thus, the life of the bio-probe assembly can be extended and its cost can be reduced.

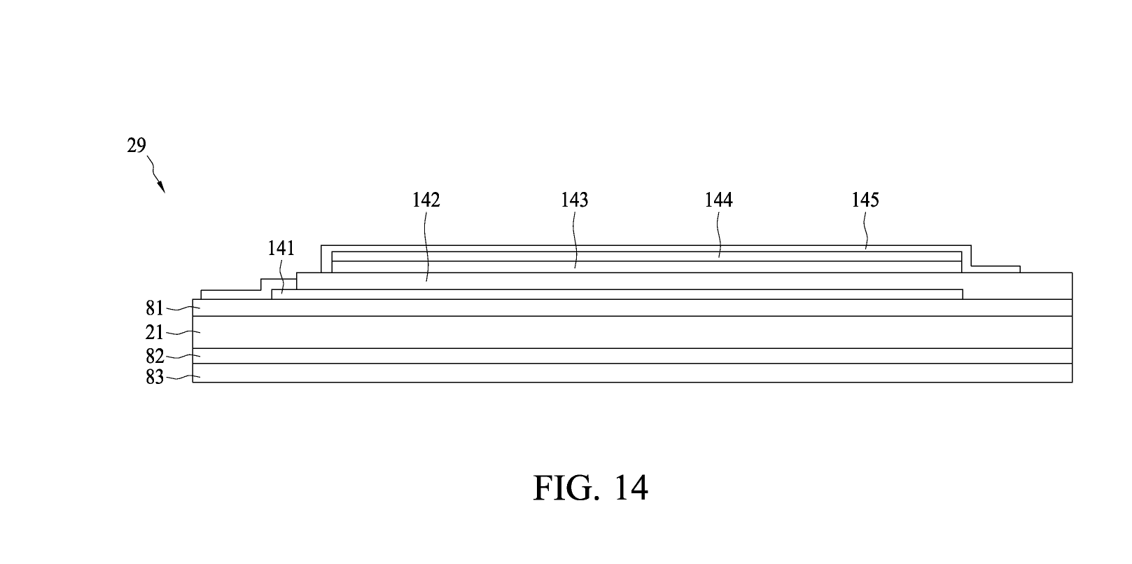

FIG. 14 schematically shows a thin film capacitor 29 according to one embodiment of the present invention. As show in FIG. 14, the thin film capacitor 29 may comprise a lower electrode 141, an upper electrode 145, and a dielectric layer 142. The lower electrode 141 may comprise polysilicon including p-type or n-type impurities. The upper electrode 145 may comprise a chrome layer 143, a nickel layer 144 and a gold layer 145. The dielectric layer 142 may comprise silicon dioxide or other insulation material.

Although the present invention and its advantages have been described in detail, it should be understood that various changes, substitutions and alterations could be made herein without departing from the spirit and scope of the invention as defined by the appended claims. For example, many of the processes discussed above can be implemented in different methodologies and replaced by other processes, or a combination thereof.

Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A bio-probe assembly comprising:
a flexible substrate comprising a surface;
a probe array comprising a plurality of probes arranged on the surface of the flexible substrate, each probe comprising a protrusion length in a range from 100 to 300 micrometers; and
an adhesive disposed on the surface of the flexible substrate.

2. The bio-probe assembly of claim 1, further comprising a circuit formed on the flexible substrate opposite to the surface, wherein the probe array electrically connects to the circuit.

3. The bio-probe assembly of claim 2, wherein the circuit comprises an antenna.

4. The bio-probe assembly of claim 1, which is configured to use an RFID, ZIGBEE, or BLUTOOTH protocol to communicate.

5. The bio-probe assembly of claim 1, wherein each probe comprises a plurality of stacking segments.

6. The bio-probe assembly of claim 1, wherein each probe comprises a mixture of polymer material and silver, or comprises an electrically conductive adhesive.

7. The bio-probe assembly of claim 1, further comprising a double-sided tape, which comprises the adhesive.

8. The bio-probe assembly of claim 1, wherein the adhesive covers the surface of the flexible substrate, except for the area where the probe array is located.

9. The bio-probe assembly of claim 1, wherein the adhesive comprises a removable adhesive.

10. The bio-probe assembly of claim 1, which uses the probe array to detect electrical impedance or potential of an acupuncture point.

11. A bio-probe assembly comprising:
a flexible substrate comprising a surface;
a first adhesive;
a probe array comprising a plurality of probes, wherein the first adhesive fastens the probe array on the surface of the flexible substrate, and each probe comprises a protrusion length in a range from 100 to 300 micrometers; and
a second adhesive disposed on the surface of the flexible substrate.

12. The bio-probe assembly of claim 11, further comprising a circuit formed on the flexible substrate opposite to the surface, wherein the probe array electrically connects to the circuit.

13. The bio-probe assembly of claim 12, wherein the circuit comprises an antenna.

14. The bio-probe assembly of claim 11, which is configured to use an RFID, ZIGBEE, or BLUTOOTH protocol to communicate.

15. The bio-probe assembly of claim 11, wherein each probe comprises a plurality of stacking segments.

16. The bio-probe assembly of claim 11, wherein each probe comprises a mixture of polymer material and silver, or comprises an electrically conductive adhesive.

17. The bio-probe assembly of claim 11, wherein the first adhesive is electrically conductive.

18. The bio-probe assembly of claim 11, wherein the first adhesive comprises a removable adhesive.

19. The bio-probe assembly of claim 11, further comprising an electrically conductive tape, which comprises an electrically conductive adhesive, wherein the probe arrays are attached to the electrically conductive tape and the first adhesive is the electrically conductive adhesive.

20. The bio-probe assembly of claim 11, further comprising a double sided tape, which comprises the second adhesive.

21. The bio-probe assembly of claim 11, wherein the second adhesive comprises a removable adhesive.

22. The bio-probe assembly of claim 11, wherein the second adhesive covers the surface of the flexible substrate, except for the area where the probe array is located.

23. The bio-probe assembly of claim 11, which uses the probe array to detect electrical impedance or potential of an acupuncture point.
